(19) United States
(12) Patent Application Publication
(10) Pub. No.: US 2017/0248581 A1
(43) Pub. Date: Aug. 31, 2017

(54) Mesenchymal Stem Cell Diagnostic Testing

(71) Applicant: NuTech Medical, Inc., Birmingham, AL (US)

(72) Inventors: Jeremy B. Vines, Birmingham, AL (US); Howard P. Walthall, Jr., Birmingham, AL (US)

(21) Appl. No.: 15/517,305

(22) PCT Filed: Oct. 7, 2015
(86) PCT No.: PCT/US2015/054463

§ 371 Date: Apr. 6, 2017

Related U.S. Application Data
(60) Provisional application No. 62/060,790, filed on Oct. 7, 2014.

(51) Int. Cl. G01N 33/50; G01N 33/74; G01N 33/569

(52) U.S. Cl. CPC G01N 33/5073; G01N 33/56966; G01N 33/74; G01N 2800/52

(57) ABSTRACT

Diagnostic tests and methods to assess the characteristics of a patient's native stem cell populations. A reference cell source may be selected for testing, to determine the relative health and potency of the cell population derived from that cell source based on standardized testing protocols, and to use the results to evaluate the systemic stem cell health status of the patient across the many different cell populations residing in different tissues in the body.

### RELATED APPLICATION

This application claims priority to U.S. Provisional Patent Application 62/060,790, filed on Oct. 7, 2014, titled “Mesenchymal Stem Cell Diagnostic Testing,” the entire contents of which are incorporated herein by reference.

### TECHNICAL FIELD

The present invention relates to diagnostic testing systems and methods to assess the characteristics of a patient's native stem cell populations.

### BACKGROUND OF THE INVENTION

Stem cells are found in all multicellular organisms and have the potential to develop into a multitude of cell types during early life and growth. Stem cells are characterized by their ability for self-renewal (i.e., maintaining their undifferentiated state during several rounds of cell division), and their potency (i.e., the ability to differentiate into specialized cell types). An adult stem cell is defined as an undifferentiated cell, found among differentiated cells in a tissue or organ, capable of renewing itself and differentiating to yield some or all of the major specialized cell types of the tissue or organ. The primary roles of adult stem cells in a living organism are to maintain and repair the tissue in which they are found. Scientists also use the term somatic stem cell instead of adult stem cell, where somatic refers to cells of the body (not the germ cells, sperm or eggs). Embryonic stem cells are defined by their origin (i.e., cells from the preimplantation-stage embryo).

Cell potency is a general term that describes the stem cell's ability to differentiate into different cell types. The more cell types a stem cell can differentiate into, the greater its potency. Totipotency is the ability of a single cell, for example spores and zygotes, to divide and produce all of the differentiated cells in an organism. Totipotent cells are those with the greatest differentiation potential. Pluripotency refers to a stem cell that has the potential to differentiate into cells representative of any of the three germ layers: endoderm, mesoderm, or ectoderm (epidermal tissues and nervous system). Multipotency describes progenitor cells that have the gene activation potential to differentiate into multiple, but limited, cell types. Oligopotency is the ability of progenitor cells to differentiate into a few cell types. Finally, a unipotent cell is a stem cell that has the capacity to differentiate into only one cell type.

The potency of a stem cell population can also be described in other ways, based on the relative capabilities of that population to perform typical stem cell functions such as proliferation, migration, attachment, engraftment, and cellular communication via the production of bioactive proteins and other signaling molecules.

In the realm of allogeneic stem cell therapies for regenerative applications, one of the most recognized and studied form of cells are mesenchymal stem cells (MSCs). These cells are generally multipotent and reside in diverse host tissues. They were first isolated from bone marrow (BM) and the stroma of spleen and thymus. Subsequently BM aspirates was considered to be the most accessible and enriched source of MSCs. Since then, MSCs have being isolated from various sites including fat, cartilage, periostium, synovium, synovial fluid, muscle and tendons. Fetal tissue, placenta, umbilical blood and vasculature have been also reported to contain MSCs (Pountos I., et al. 2005).

A significant amount of research has been conducted over the last two decades confirming the central importance of MSCs in the regeneration of various soft tissues and bone. MSCs have been associated with key healing processes including the down-regulation of the initial inflammation resulting from tissue damage, the recruitment of cell types critical to healing from the surrounding tissues, matrix deposition, organization and morphogenesis, and the development of circulatory support for the regenerated tissue.

Other research efforts have evaluated the potential therapeutic benefit of both autologous (patient-derived) and allogeneic (donor-derived) MSCs in the treatment of various pathological conditions, including systemic conditions such as graft versus host disease and focal soft tissue injuries. In general, these efforts have met with at least modest success (Bobis et al. 2006; Bernardo and Fibbe 2012). However, it has increasingly been recognized that the efficacy of cellular therapy depends in part on the health and age of the donor from whom the cells are isolated, which may affect the therapeutic potency of the cells recovered. Likewise, part of the difference in native healing potential between patients affected with similar conditions or following similar surgeries may be explained by the potency of their native stem cell populations.

In general, as people age, both the density and regenerative potential of their endogenous stem cell population declines (Caplan 1994; Zhou, et al. 2008; Zaim, et al. 2012). Unhealthy behavioral patterns, harsh environmental conditions, and systemic pathologies all likely influence the health status of native stem cell populations. Diabetes, heart disease, obesity, smoking, and alcohol and drug abuse in particular are all suspected of having such effects. These are not the only factors in play, however, as individual variation and genetics likely also play a role. Several researchers have worked with cells intended for potential allogeneic transplantation to assess these effects, and in a few cases tests have been proposed for use in the evaluation of allogeneic stem cell populations prior to transplantation. (Russell, et al. (2011); Deskins, et al. (2013); Janicki, et al (2011)). No consensus on this subject has emerged, however, perhaps in part because of the wide array of functions performed by MSCs in various different tissues in vivo. Furthermore, use of such tests as a diagnostic tool to assess native MSC populations has not been emphasized, perhaps in part due to the difficulty of accessing many of the relevant tissue types absent surgical intervention. For example, the pain and morbidity associated with bone marrow biopsies may render use of a pre-treatment test requiring culturing of bone marrow cells impracticable. Other approaches involving the use of radiologic and other testing not directly involving stem cell fitness have been suggested, but have not been shown to be useful outside narrow disease areas and have not been widely adopted. (Kim, et al (2009))

Despite the intense research interest in the transplantation of MSCs, the diagnostic evaluation of native MSC cell populations remains elusive and underserved.

### SUMMARY OF THE INVENTION

The health of the native populations of MSCs throughout a patient's body should be closely correlated in the absence of a localized disease condition. Effects such as aging, smoking, vascular and cardiovascular conditions, diabetes, and other health conditions known to weaken the effectiveness of MSCs have been demonstrated to affect multiple stem cell populations throughout the body, as should be expected given the systemic nature of these conditions (Zaim, et al. 2012; Zhou, et al. 2008; Efimenko, et al (2014); Stolzing, et al. (2008)). For this reason it is possible to select a Reference Cell Source for testing, to determine the relative health and potency of the cell population derived from that cell source based on standardized testing protocols, and to use the results to evaluate the systemic MSC health status of the patient across the many different cell populations residing in different tissues in the body.

The selection of a Reference Cell Source should be made with ease of recovery in mind. As used herein, “Reference Cell Source” may be peripheral blood, superficial fat, skin, or oral or nasal mucosa.

Multiple tests have been proposed to evaluate the health and potency of MSCs. These include the number of MSCs identified per a set tissue weight, the presence of certain cell surface markers, the presence of certain proteins inside the cells, the presence of RNA indicating the transcription of certain genes, telomere length, telomerase activity, methylation status, cellular proliferation, migration, and/or differentiation, and the rate of production of certain growth factors or other signaling molecules. In alternative embodiments of the present invention, the diagnostic testing method could include any one or more of these tests, or other tests of stem cell potency known in the art.

### DETAILED DESCRIPTION

Before the present compositions, articles, devices, and/or methods are disclosed and described, it is to be understood that they are not limited to specific methods unless otherwise specified, or to particular reagents unless otherwise specified, and as such may vary. It is also to be understood that the terminology as used herein is used only for the purpose of describing particular embodiments and is not intended to be limiting.

This application references various publications. The disclosures of these publications, in their entireties, are hereby incorporated by reference into this application to describe more fully the state of the art to which this application pertains. The references disclosed are also individually and specifically incorporated herein by reference for material contained within them that is discussed in the sentence in which the reference is relied on.

The term “mammalian” as used herein, encompasses any mammal, for instance a human. A “stem cell” is a cell which has the potential to differentiate into multiple different cell types, and includes both multipotent and pluripotent cells. As used herein, the term “treat” refers to any type of treatment which imparts a benefit to a mammalian subject in need thereof “Treat” may also refer to the alleviation of one or more symptoms. A “subject”, as used herein, is any mammalian subject and may include a patient or individual in need of therapy or treatment. A “biologically compatible solution” refers to a synthetic or natural solution which may be placed in intimate contact with living tissue without damage to such tissue. A biologically compatible solution has compositions and properties similar to solutions made by a living organism and thus will not harm the organism or cause adverse reactions within the organism. A preparation including a biologically compatible solution combined with the prenatal stem cells, stem cell populations, or secretions of the present invention may be used for the parenteral administration into a subject to treat a specific condition. Such administration may be done intravenously, intramuscularly, subcutaneously or through implantation.

The diagnostic testing of native MSC health status disclosed herein provides insight into the capability of a patient to heal a wide variety of medical conditions, and thus improves medical decision-making. For example, the MSC health status determination contemplated herein could indicate whether a patient should be able to heal an injury without support from transplanted MSCs, whether autologous MSCs could be an adequate treatment, or whether the use of allogeneic MSCs is appropriate due to the poor health condition of the patient's own MSC populations. MSC health status could also assist in determining whether a patient may be capable of recovering from a demanding surgical procedure, or whether a less invasive procedure may be preferred. MSC health status may also help set realistic expectations about the likely course of a patient's recovery and improve post-operative recovery planning and allow comparison against known ranges from patients with differing healing capacities. To allow such assessment, a diagnostic testing method is disclosed that allows ready comparison between patients against a standardized scale.

In one embodiment, tissue from a readily accessible and pre-determined Reference Cell Source is collected from mammalian subjects requiring treatment for a condition. The Reference Cell Source may be peripheral blood, superficial fat, skin, or oral or nasal mucosa. Peripheral blood may be drawn from a patient and collected using a blood collection tube. Blood collection tubes are known in the art and may include cell separation material, such as a gel, and/or anti-coagulants, such as EDTA and heparin. Preferably, if skin, fat, or mucosa is used, the sample should be obtained from the same anatomical area in all patients, e.g. a skin biopsy from the upper arm. Stem cells may be isolated from these various tissue sources according to techniques known in the art, and then cultured using a standardized culture methodology. (E.g. Efimenko, et al. (2014); Stolzing, et al (2008); Ab Kadir, et al. (2012); Russell, et al. (2011); Manini, et al (2011); Lermen, et al (2010)). The stem cell population, selected for as described above, may be harvested or collected in an appropriate Cell Propagation Medium. As used herein, “Cell Propagation Medium” include media such as Hank's Balanced Salt Solution (HBSS), RPMI, Dulbecco's Modified Eagle Medium (DMEM), Iscove's modified Dulbecco's medium (IMDM) or Dulbecco's phosphate buffered saline (dPBS). The Cell Propagation Medium may be Supplemented. As used herein, “Supplemented” means the inclusion of one or more of fetal calf serum (FCS), fetal bovine serum (FBS), bovine serum albumin (BSA), human serum albumin (HSA), recombinant human albumin (RHA), HEPES buffer, Insulin, Transferrin, Selenium, and other cell culture supplements known in the art. The Cell Propagation Medium allows for the growth of the cells under standardized, controlled conditions. Additionally, to maintain their endogenous state and promote healthy culture, the cells may be propagated on a substrate consisting of either natural extracellular matrix proteins or synthetic derivatives such as Collagen, Fibronectin, Laminin or a synthetic peptide coating. The enriched cell populations may then be propagated to expand their numbers. The term “propagated”, as defined herein, refers to increasing the number of viable cells in a particular culture, typically by growing the cells through one or more cell cycles.

After a predetermined period of cell culturing and growth, the resulting cells may then be tested for potency using one or more of several potency testing methods. The results may then be combined and reported using a standardized reporting scale. As will be understood by one skilled in the art, various test results may be combined in weighted fashion to control for differences in orders of magnitude and to adjust the relative influence of each test on the final calculated number. Further, the tests included in the standardized index, and the index itself, may be validated against other types of tests using statistical methods well known in the art to establish the correlation between the standardized index and other aspects of stem cell behavior.

The final standardized result may be compared against the results for other subjects and used in the evaluation of treatment options for the subject, e.g. the identification of disease conditions, the selection of surgery or other treatments, and the determination of whether autologous, allogeneic, or other cell or biologic supplementation is appropriate in a particular case. Importantly, the results may be used in the treatment of the patient relating to conditions involving tissue types other than those involved in the tests. The results may also be used in the context of clinical trials to evaluate the effect of stem cell health on the efficacy of certain treatments or products.

### Stem Cell Markers

In one embodiment, the cells may be sorted based upon the expression of markers, such as through fluorescent activated cell sorting (FACS) or magnetic activated cell sorting (MACS). The cells isolated from the tissue sample may be selected for the presence of a particular marker, for instance surface markers, intracellular markers or secreted proteins. These cells may also be sorted and/or counted by cell sorting techniques utilizing antibodies binding to the marker, and the relative number of such cells may be counted and compared against the number of cells not testing positive for such markers.

For instance, stage specific embryonic antigens (SSEAs) are a group of glycolipid carbohydrate epitopes. One such antigen, SSEA-4, is expressed upon the surface of human teratocarcinoma stem cells (EC), human embryonic germ cells (EG) and human embryonic stem cells (ES). Expression of SSEA-4 is down regulated following differentiation of human EC cells. In contrast, the differentiation of murine EC and ES cells may be accompanied by an increase in SSEA-4 expression. SSEA-4 is thus considered by some researchers to be a marker for a stem cell of high potency, even pluripotency.

CD105 (commonly referred to as Endoglin, END, F1141744, HHT1, ORW and ORW1) is a type I membrane glycoprotein located on cell surfaces and is part of the TGF-beta receptor complex. CD105 plays a crucial role in angiogenesis. In adult cell populations, the presence of CD105 is considered to be a standard marker of a therapeutically-effective MSC (Dominici, et al. 2006).

CD44 is a cell-surface glycoprotein involved in cell-cell interactions, cell adhesion, and migration is expressed on both MSCs and amniotic fluid-derived stem cells (Roubelakis, et al. 2007).

C-kit (also known as CD117 or tyrosine-protein kinase Kit) is a protein encoded in humans by the KIT gene. Multiple transcript variants encoding different isoforms have been found for this gene. C-kit is a mast/stem cell growth factor receptor that is known to be present on certain types of mesenchymal stem cells. C-kit positive amniotic fluid derived cells have been demonstrated to have a higher affinity to differentiate into different lineages than c-kit negative cells (Arnhold, et al. 2011; Bai, et al. 2012). A number of researchers have suggested the use of c-kit positivity as a marker for selection of a therapeutically-beneficial cell population (De Coppi, et al. 2007; Pozzobon, et al. 2013).

In certain embodiments, the marker antibodies (e.g. SSEA-4, CD105, CD44 and/or c-kit antibodies) may be conjugated with certain molecules, such as a label, to assist in the identification and separation of the desired stem cells. As used herein, “label” may include, but is not limited to, fluorescein isothiocyanate (FITC), phycoerythrin (PE), Cy5PE, Cy7PE, Texas Red (TR), allophycocyanin (APC), Cy5, Cy7APC, Cascade Blue, biotin, avidin and streptavidin. The antibodies are generally added to a cell sample in a concentration sufficient to allow for binding to the cell or cell population of interest, as known to one of skill in the art. The antibody and cells are incubated so that complexes are formed.

The use of such cell surface antigens or other markers provides a means of selecting for and/or counting particular populations. For example, cells may be selected for by flow cytometry utilizing a conjugated CD105 antibody (i.e., using FACS or MACS). Other immune-selection methods, for instance those utilizing solid phase chromatography, are also contemplated. One of skill in the art will appreciate that many techniques may be employed for the immune-separation and counting of the desired cells. In one embodiment, a ratio of selected cells to total cells may then be calculated and used as part of a standardized index of cell potency.

### Cell Viability and Proliferation

The viability and proliferation of the cells may be measured utilizing techniques well known in the art. Cell viability may be measured by staining the cells with various dyes. Tools for measuring cell proliferation include probes for analyzing the average DNA content and cellular metabolism in a population, as well as single-cell indicators of DNA synthesis and cell cycle-specific proteins. In some embodiments, the cells are proliferated through one to ten or more passages, and the number of cells assessed at predetermined time points to assess the rapidity of cell proliferation and of population growth.

For example, cells from the mammalian subject may be seeded on 48 well tissue culture plates (BD Biosciences, San Jose, Calif.) at a density of˜10,000 cells/cm² in replicates of six and allowed to incubate for 7 days. Cells may then be collected at days 1, 4, and 7 and quantified utilizing a Picogreen dsDNA assay (Invitrogen, Carlsbad, Calif.). To determine the amount of cells present in each sample, cells may be determined to have a particular quantity of DNA per cell or a sample curve may be made using a known number of cells. To compare cell morphology and density over time, cells may be stained with Phalloidin (Invitrogen, Carlsbad, Calif.) and 4′,6-diamidino-2-phenylindole (DAPI) (Invitrogen, Carlsbad, Calif.) and imaged under fluorescence with a microscope.

Cell proliferation may also be quantified via the detection of 5-bromo-2′-deoxyuridine (BrdU), which is incorporated into cellular DNA during cell proliferation and may be quantified using an anti-BrdU antibody.

In certain embodiments, the stem cells or cell population may be propagated in the presence of a reagent capable of suppressing differentiation. Such reagents are known in the art and include leukemia inhibitory factor, stem cell factor and certain metal ions. In an additional embodiment, reagents may be added to the prenatal stem cells or cell population to induce differentiation, for example Ca⁺, hydrocortisone, keratinocyte growth factor and collagen. For example, the ability of cells to differentiate along osteogenic, chondrogenic, adipogenic and other tissue lineages may be tested using techniques known in the art (e.g. Peister, et al. 2011). Likewise, the ability of cells plated at standard densities to form clonal colonies may be evaluated via a colony forming unit (CFU-F) assay (E.g. Stolzing, et al (2008)).

The cell viability and/or rate of cell proliferation and/or CFU results may be determined as described herein and used as part of a standardized index of cell potency. Likewise various indicators of differentiation may be quantified and included in the standardized index.

### Growth Factor and Protein Production

The propagated stem cells may secrete various hormones, enzymes, growth factors, etc., that are believed to be associated with the effective function of these cells in vivo. (Efimenko, et al (2014)). The amount of such factors produced in a given time may be determined by ELISA or other means. For example, the levels of production of Hepatocyte growth factor (HGF), Insulin-like Growth Factor (IGF), Vascular Endothelial Growth Factor (VEGF), Transforming Growth Factor Beta 1 and Beta 3 (TGF-β1 and TGF-β3), Angiogenin (ANG), Angiopoieten 2 (ANGPT-2). The production levels of one or more of these growth factors may be included in the standardized index. Further, the growth factor production levels may or may not be normalized based on the number of cells present to eliminate the effect of cell proliferation rate.

The production of alkaline phosphatase (ALP) may also be tested using techniques known in the art, either in neutral culture or in osteogenic differentiation media. The production of ALP is an indicator of stem cell activity and robustness, and the level of production may be used as part of the standardized index described herein.

Conversely, the levels of certain markers associated with aging may be measured, with higher levels tending to indicate poor stem cell capabilities. (Stolzing, et al (2008), Russell, et al (2011)). These levels may be used in calculating the standardized index described herein.

### In Vitro and In Vivo Tests

Various standardized in vitro or in vivo tests may also be utilized. For example, standardized in vitro models used in the art include Boyden chamber cell migration, wound healing scratch assay and endothelial cell (HUVEC, or others) tube formation angiogenesis tests. Standardized in vivo models include ectopic bone formation and full thickness wound healing with polyvinyl alcohol sponges. (See, e.g., Efimenko, et al (2014), Deskins, et al (2013), Janicki, et al (2011); Kim, et al (2009)) These tests, particularly in vivo tests, are typically more expensive and may require more resources and time to conduct than other testing approaches. However, if desired the results of these tests may also be used in calculating the standardized index described herein.

### Example Embodiment

In an exemplary embodiment, the tests to be done may be so arranged and combined to minimize the amount of work done and reduce the number of quantitative measurements and specialized measuring equipment required. In one exemplary embodiment, a sample of venous peripheral vascular blood may be taken from a mammalian subject using standard techniques. In other embodiments small samples of skin or subcutaneous fat may be taken. The stem cells from the sampled tissue may be isolated and cultured using techniques known in the art. After being grown in culture, with media changes every three days, for seven days, or to confluence if that occurs earlier than seven days, plastic-adherent cells may be plated in 48 well tissue culture plates (BD Biosciences, San Jose, Calif.) in culture media without FBS at a density of˜100,000 cells/cm² and allowed to incubate for three (3), or alternatively four (4) or seven (7) days. Cell plating density may be determined using a handheld cell counter such as the Scepter Handheld Automated Cell Counter (Millipore). Supernatants or whole cell lysates may then be collected and assessed for Growth Factor content utilizing ELISA, or preferably a quantitative multiplex assay (Ray Biotech). As used herein, the term “Growth Factor” includes one or more growth factors, including, but not limited to, HGF, IGF-I, IGF-II, VEGF, ANGPT-2,ANG, TGF β3, TGF β1, Tumor necrosis factor-inducible gene 6 (TSG-6), and soluble IGF-II/Mannose 6 Phosphate Receptor (sIGF-II/MPR). Growth Factor levels at this time point will reflect the amount of growth factors produced per cell, as well as the effects of cellular proliferation rates during the culture period. The quantification of Growth Factors may be more consistent than conventional cell viability and proliferation assay methods. (Deskins, et al (2013)) The Growth Factors referenced are known to be involved in angiogenesis and/or immune modulation, important stem cell functions which are applicable across multiple tissue types. (Efimenko, et al (2014); Kim, et al (2009); Lee, et al (2009), Watt, et al (2013)). The total level of the selected Growth Factors may then be used as the standardized index described herein, or combined with other test results if desired.

### REFERENCES

Ab Kadir, Ruzanna, et al. (2012). “Characterization of Mononucleated Human Peripheral Blood Cells.” The Scientific World Journal Vo. 2012, Article ID 843843
Arnhold, S., S. Gluer, et al. (2011). “Amniotic-Fluid Stem Cells: Growth Dynamics and Differentiation Potential after a CD-117-Based Selection Procedure.” Stem Cells Int 2011: 715341.
Bai, J., Y. Wang, et al. (2012). “Human amniotic fluid-derived c-kit(+) and c-kit (−) stem cells: growth characteristics and some differentiation potential capacities comparison.” Cytotechnology 64(5): 577-589.
Bernardo, M. E. and W. E. Fibbe (2012). “Safety and efficacy of mesenchymal stromal cell therapy in autoimmune disorders.” Ann N Y Acad Sci 1266: 107-117.
Bobis, S., D. Jarocha, et al. (2006). “Mesenchymal stem cells: characteristics and clinical applications.” Folia Histochem Cytobiol 44(4): 215-230.
Caplan, A. I. (1994). “The mesengenic process.” Clin Plast Surg 21(3): 429-435.
De Coppi, P., G. Bartsch, Jr., et al. (2007). “Isolation of amniotic stem cell lines with potential for therapy.” Nat Biotechnol 25(1): 100-106.
Delo, D. M., P. De Coppi, et al. (2006). “Amniotic fluid and placental stem cells.” Methods Enzymol 419: 426-438.
Diaz-Prado SM, et al. Cell Tissue Bank 11, 183-195 (2010)
Deskins, Desirae L., et al. (2013). “Human Mesenchymal Stromal Cells: Identifying Assays to Predict Potency for Therapeutic Selection.” Stem cells Translational Medicine 2013; 2:151-158
Dominici, M., Le Blanc, K., et al. (2006). “Minimal criteria for defining multipotent mesenchymal stromal cells. The International Society for Cellular Therapy position statement.” Cytotherapy 8(4): 315-317.
Efimenko, Anastasia, et al. (2014). “Adipose-Derived Mesenchymal Stromal Cells From Aged Patients With Coronary Artery Disease Keep Mesenchymal Stromal Cells Properties but Exhibit Characteristics of Aging and Have Impaired Aniogenic Potential.” Stem Cells Translational Medicine 3:32-41
Janicki, Patricia, et al. (2011). “Prediction of In Vivo Bone Forming Potency of Bone Marrow-Derived Human Mesenchymal Stem Cells.” European Cells and materials Vol. 21 2011 (pages 488-507)
Kim, Byung-Hak, et al. (2009). “The Effectiveness of Bone Mineral Density as Supplementary Tool for Evaluation of the Osteogenic Potential in Patients with Spinal Fusion.” Asian Spine Journal Vol. 3, No. 1, pp. 1-9
Lee, Ryang Hwa, et al. (2009). “Intravenous hMSCs Improve Myocardial Infarction in Mice Because Cells Embolize in Lung Are Activated to Secret the Anti-inflammatory Protein TSG-6.” Cell Stem Cell 5, 54-64
Lermen, Dominik, et al. (2010). “Neuro-Muscular Differentiation of Adult Porcine Skin Derived Stem cell-like cells.” PLoS ONE (2010) Vol. 5, Issue 1, e8968
Manini, Ivana, et al. (2011). “Multi-potent progenitors in freshly isolated and cultured human mesenchymal stem cells: a comparison between adipose and dermal tissue.” Cell Tissue Res (2011) 344:85-95
Okano, H., M. Nakamura, et al. (2013). “Steps toward safe cell therapy using induced pluripotent stem cells.” Circ Res 112(3): 523-533.
Peister, A., M. A. Woodruff, et al. (2011). “Cell sourcing for bone tissue engineering: amniotic fluid stem cells have a delayed, robust differentiation compared to mesenchymal stem cells.” Stem Cell Res 7(1): 17-27.
Pozzobon, M., Piccoli, M. et al. (2013). “Isolation of c-Kit+human amniotic fluid stem cells from second trimester.” Methods Mol Bio 1035:191-198.
Pountos I., et al. (2005). “Biology of mesenchymal stem cells.” Injury, Int J Care Injured 36S: S8-S12.
Roubelakis, M. G., K. I. Pappa, et al. (2007). “Molecular and proteomic characterization of human mesenchymal stem cells derived from amniotic fluid: comparison to bone marrow mesenchymal stem cells.” Stem Cells Dev 16(6): 931-952.
Roubelakis, M. G., 0. Trohatou, et al. (2012). “Amniotic fluid and amniotic membrane stem cells: marker discovery.” Stem Cells Int 2012: 107836.
Russell, Katie, C., et al. (2011). “Clonal Analysis of he Proliferation Potential of Human Bone Maroow Mesenchymal Stems Cells as a Finction of Potency.” Biotechnol Bioeng 108(11):2716-2726
Stolzing, A., et al. (2008). “Agre-related changes in human bone marrow-derived mesenchymal stem cells: Consequences for cell therapies.” Mechanisisms of Ageing and Develeopment 129 (2008) 163-173
Watt, Suzanne M., et al. (2013). “The angiogenic properties of mesenchymal stem/stromal cells and their therapeutic potential.” British Medical Bulletin 2013; 108: 25-53
Zaim, M., S. Karaman, et al. (2012). “Donor age and long-term culture affect differentiation and proliferation of human bone marrow mesenchymal stem cells.” Ann Hematol 91(8): 1175-1186.
Zhou, S., J. S. Greenberger, et al. (2008). “Age-related intrinsic changes in human bone-marrow-derived mesenchymal stem cells and their differentiation to osteoblasts.” Aging Cell 7(3): 335-343.

1. A method to assess the characteristics of a patient's native stem cell populations, comprising the steps of:
obtaining a Reference Cell Source from a patient;
isolating stem cells from the Referenced Cell Source;
culturing the isolated stem cells in a Cell Propagation Medium;
measuring the potency of the isolated stem cells using one or more techniques; calculating an index value using the outcomes of such potency measurements; and
determining an assessment value for the patient's isolated stem cells based on a comparison of their index value to a range of values determined from other subjects using the same standardized techniques.

2. The method of claim 1, wherein the Reference Cell Source is peripheral blood.

3. The method of claim 1, wherein the Reference Cell Source is superficial fat.

4. The method of claim 1, wherein the Reference Cell Source is skin.

5. The method of claim 1, wherein the Reference Cell Source is nasal mucosa.

6. The method of claim 1, wherein the stem cells are sorted using FACS.

7. The method of claim 1, wherein the stem cells are sorted using MACS.

8. The method of claim 1, wherein the Cell Propagation Medium is Hanks Balanced Salt Solution.

9. The method of claim 1, wherein the Cell Propagation Medium is RPMI.

10. The method of claim 1, wherein the Cell Propagation Medium is Dulbecco's Modified Eagle Medium.

11. The method of claim 1, wherein the Cell Propagation Medium is Iscove's modified Dulbecco's medium.

12. The method of claim 1, wherein the Cell Propagation Medium is Dulbecco's phosphate buffered saline.

13. The method of claim 1, wherein the Cell Propagation Medium is Supplemented.

14. The method of claim 1, wherein the stem cells are recultured.

15. The method of claim 14, wherein the potency of the stem cells is determined by measuring the concentrations of Growth Factors in the stem cell culture supernatants.

16. The method of claim 14, wherein the potency of the stem cells is determined by measuring the concentrations of Growth Factors in the stem cell lysates.

17. The method of claim 1, wherein the potency of the stem cells is determined by measuring their ability to differentiate into a particular cell type.

18. The method of claim 1, wherein the potency of the stem cells is determined by measuring the ratio of the stem cells to the total cells of the Reference Cell Source.

19. The method of claim 1, wherein the potency of the stem cells is determined by measuring the rate at which said cells proliferate.

20. The method of claim 1, wherein the potency of the stem cells is determined by determining the relative presence of certain surface or internal markers.

21. The method of claim 1, wherein the relationship between the index value for the patient's stem cells and the range of values determined from other subjects is used in determining the appropriate treatment of the patient.

22. A kit for assessing the characteristics of a patient's native stem cell populations comprising:
at least one blood collection tube;
at least one stem cell-specific antibody, wherein each antibody conjugated to a label;
a Cell Propagation Medium;
at least one Supplement; and
at least one Growth Factor-specific antibody, wherein each antibody conjugated to a label.
